(19) United States
(12) Patent Application Publication
(10) Pub. No.: US 2023/0118748 A1
(43) Pub. Date: Apr. 20, 2023

(54) PROSTHETIC CARDIAC VALVE DEVICES, SYSTEMS, AND METHODS

(71) Applicant: SHIFAMED HOLDINGS, LLC, Campbell, CA (US)

(72) Inventor: Claudio ARGENTO, Felton, CA (US)

(21) Appl. No.: 17/906,216

(22) PCT Filed: Mar. 10, 2021
(86) PCT No.: PCT/US2021/021647

§ 371 Date: Sep. 13, 2022

Related U.S. Application Data
(60) Provisional application No. 62/989,428, filed on Mar. 13, 2020.
(60) Provisional application No. 62/992,685, filed on Mar. 20, 2020.

(51) Int. Cl. A61F 2/24

(52) U.S. Cl. CPC A61F 2/2418; A61F 2/2436; A61F 2220/0008

(57) ABSTRACT

A system for treating a diseased native valve in a patient, the system comprising a compressible and expandable frame structure with an expanded configuration comprising one or more anchor retaining elements disposed along one or more lateral sides of the frame structure, and one or more anchors having a free end. In one embodiment, the anchors are slidably disposed within the one or more anchor retaining elements of the frame structure and can be configured to be releasably attached to one or more proximal anchor pusher elements disposed within an inner shaft of a delivery device.

### CROSS REFERENCE TO RELATED APPLICATIONS

This application claims priority to U.S. Provisional Application No. 62/989,428, filed Mar. 13, 2020, entitled “PROSTHETIC CARDIAC VALVE DEVICES, SYSTEMS, AND METHODS”; and U.S. Provisional Application No. 62/992,685, filed Mar. 20, 2020, entitled “PROSTHETIC CARDIAC VALVE DEVICES, SYSTEMS, AND METHODS,” each of which is herein incorporated by reference in its entirety.

This application may also be related to U.S. patent application Ser. No. 16/546,901, filed Aug. 21, 2019, published as U.S. Patent Application Publication No. US 2020-0060852 A1, entitled “PROSTHETIC CARDIAC VALVE DEVICES, SYSTEMS, AND METHODS”; U.S. patent application Ser. No. 16/594,946, filed Oct. 7, 2019, issued as U.S. Pat. No. 10,912,644, entitled “PROSTHETIC CARDIAC VALVE DEVICES, SYSTEMS, AND METHODS”; U.S. patent application Ser. No. 16/723,537, filed Dec. 20, 2019, published as U.S. Patent Publication No. US 2020-0261220 A1, entitled “PROSTHETIC CARDIAC VALVE DEVICES, SYSTEMS, AND METHODS”; International Patent Application No. PCT/US2019/047542, filed Aug. 21, 2019, published as WO 2020/041495, entitled “PROSTHETIC CARDIAC VALVE DEVICES, SYSTEMS, AND METHODS”; International Patent Application No. PCT/US2019/055049, filed Oct. 7, 2019, published as WO 2020/073050, entitled “PROSTHETIC CARDIAC VALVE DEVICES, SYSTEMS, AND METHODS”; International Patent Application No. PCT/US2019/057082, filed Oct. 18, 2019, published as WO 2020/082039, entitled “ADJUSTABLE MEDICAL DEVICE”; International Patent Application No. PCT/US2019/068088, filed Dec. 20, 2019, published as WO 2020/132590, entitled “PROSTHETIC CARDIAC VALVE DEVICES, SYSTEMS, AND METHODS”; International Patent Application No. PCT/US2020/023671, filed Mar. 19, 2020, published as WO 2020/191216, entitled “PROSTHETIC CARDIAC VALVE DEVICES, SYSTEMS, AND METHODS”; International Patent Application No. PCT/US2020/027744, filed Apr. 10, 2020, published as WO 2020/210685, entitled “MINIMAL FRAME PROSTHETIC CARDIAC VALVE DELIVERY DEVICES, SYSTEMS, AND METHODS”; International Patent Application No. PCT/US2020/058413 filed Oct. 30, 2020, entitled “PROSTHETIC CARDIAC VALVE DELIVERY DEVICES, SYSTEMS, AND METHODS”, and International Patent Application No. PCT/US2021/020704 filed Mar. 3, 2021, entitled “PROSTHETIC CARDIAC VALVE DEVICES, SYSTEMS, AND METHODS”, which are incorporated herein by reference for all purposes in their entireties.

### BACKGROUND

Expandable frame devices are useful in various medical capacities, including applications comprising stenting open biological structures and as a mechanical support during surgical procedures. In general, it is advantageous to maximize the difference in at least one dimension of an expanded configuration of an expandable frame device and its collapsed configuration so that the device can be delivered to a target location inside of a subject in as minimally invasive a manner as possible. To facilitate large differences in this at least one dimension (of an expandable frame device when the device is in an expanded configuration versus when it is in a collapsed configuration), expandable frame devices generally comprise materials and geometries suited for bending. The present disclosure generally relates to treating a diseased native valve in a patient and more particularly relates to prosthetic heart valves and their methods of delivery.

Blood flow between heart chambers is regulated by native valves—the mitral valve, the aortic valve, the pulmonary valve, and the tricuspid valve. Each of these valves are passive one-way valves which open and close in response to differential pressures. Patients with valvular disease have abnormal anatomy and/or function of at least one valve. For example, a valve may suffer from insufficiency, also referred to as regurgitation, when the valve does not fully close and allows blood to flow retrograde. Valve stenosis can cause a valve to fail to open properly. Other diseases may also lead to dysfunction of the valves. While medications may be used to treat the disease, in many cases the defective valve may need to be repaired or replaced at some point during the patient's lifetime. Existing valves and surgical repair and/or replacement procedures may have increased risks, limited lifespans, and/or are highly invasive. Some less-invasive transcatheter options are available, however these generally are limited to aortic valve procedures, are limited in their patient-to-patient flexibility, and often take longer than desired to implant.

### SUMMARY

It would therefore be desirable to provide a less invasive procedure for repair and replacement of heart valves, including the mitral valve, quicker surgical methods, and/or prosthetic valves that can accommodate a variety of individual patients. Disclosed herein are devices, systems and methods for delivering a valve prosthesis comprising a spiral anchor and a frame structure from a delivery device. Not necessarily all such aspects or advantages are achieved by any particular embodiment. Thus, various embodiments may be carried out in a manner that achieves or optimizes one advantage or group of advantages taught herein without necessarily achieving other aspects or advantages as may also be taught or suggested herein.

The present disclosure generally relates to treating a diseased native valve in a patient and more particularly relates to delivery devices, systems, and methods from delivering and deploying a valve prosthesis comprising a spiral anchor and a frame structure adjacent the diseased native valve, the spiral anchor being deployed around one or more structures of the heart and configured to anchor the frame structure to the native valve when the frame structure is expanded therein.

Disclosed herein is a system for treating a diseased native valve in a patient, the system comprising a frame structure having a compressed configuration and an expanded configuration wherein the frame structure comprises one or more anchor retaining elements dispose along one or more lateral sides of the frame structure; one or more anchor elements having a free end and configured to be slidably disposed within the one or more anchor retaining elements of the frame structure; wherein the anchor is configured to be fully disposed within the anchor retaining elements in a delivery configuration, wherein the anchor is configured to be at least partially external to the anchor retaining elements in a deployed configuration, and wherein the anchor is configured to be advanced from the delivery configuration to the deployed configuration when the frame structure is in the expanded configuration adjacent the native valve.

In some embodiments, the system can further comprise a delivery device, the delivery device comprising an outer sheath and an inner sheath extendable from the outer sheath, the inner sheath having a lumen through which proximal anchor pusher elements are threaded, wherein the proximal anchor pusher elements are connected to one or more anchors by one or more anchor releasing elements.

In some embodiments, the delivery device can further comprise a delivery guidewire advanceable through the lumen of the inner sheath. The outer sheath can have a lumen and is configured to maintain the inner sheath in an elongated configuration when positioned within the lumen. The outer sheath can be steerable. The inner sheath can be steerable.

In some embodiments, a proximal end of the one or more anchors can be detachably coupled to the one or more proximal anchor pusher elements by one or more anchor releasing elements during delivery to the native valve. The one or more anchors can be configured to be elongated when disposed within the anchor retaining elements. Advancement of the one or more anchor pusher elements from the lumen of the inner shaft into the anchor retaining elements can actuate the one or more anchors into the deployed configuration.

In some embodiments, the one or more anchors can be maintained in the elongated configuration by radial constriction from the anchor retaining elements and wherein advancement of the one or more proximal anchor pusher elements out of the lumen of the inner shaft actuates the anchor into the deployed configuration. The proximal end of the anchor can be detachably coupled to the and wherein retraction of the one or more proximal anchor pusher elements away from the proximal end of the one or more anchors detaches the one or more anchors from the one or more proximal anchor pusher elements.

In some embodiments, the one or more anchors can be configured to lock into the one or more anchor retaining elements after actuation of the one or more anchor releasing elements. The one or more anchor releasing elements can be a weak adhesive. The frame structure can be detachably coupled to the delivery device in the compressed configuration during delivery to the native valve. The expansion of the frame structure to the expanded configuration can detach the frame structure from the delivery device. The outer sheath can be steerable. The free end can comprise an atraumatic tip. The free end can comprise a ball tip. The free end can be configured for piercing tissue. The one or more anchor elements can comprise a helical wire. The one or more anchor elements can comprise a first portion comprising the helical wire or band, and a second portion. The second portion can comprise a straight wire or band. The frame structure can be configured for expanding within the native valve of the patient. The compressed configuration can be sized and dimensioned for percutaneous insertion and the expanded configuration is sized and dimensioned for implantation in the native valve of the patient.

In some embodiments, the frame structure can comprise a first and second opposite ends, the first end extending above a native valve and the second end extending below the native valve when the frame structure is anchored to the native valve. The frame structure can sit below the native valve when the frame structure is anchored to the native valve. The frame structure can comprise an expandable stent. The expanded configuration can be a generally tubular expanded shape.

In some embodiments, the frame structure can comprise an expanded outer periphery in the expanded configuration and a compressed outer periphery when subject to an external radial force in the compressed configuration, wherein the compressed outer periphery is smaller in diameter than the expanded outer periphery. The frame structure can be balloon-expandable.

The frame structure can be self-expanding. The frame structure can be maintained in the compressed configuration by radial constriction from the outer sheath and wherein advancement of the inner shaft out of the lumen of the outer sheath actuates the frame structure into the expanded configuration.

In some embodiments, the system can further comprise a valve segment within the frame structure comprising a biocompatible one-way valve. At least a portion of the valve segment can be positioned within at least a portion of the frame structure. The valve segment can comprise at least one leaflet having an inner layer and an outer layer, and wherein the frame structure is attached to the outer layer at one or more ends of the frame structure. The valve segment can comprise a plurality of leaflets.

Disclosed herein is a system for treating a diseased native valve in a patient, the system comprising: (a) a delivery device comprising: (i) an outer sheath wherein the outer sheath has a lumen, (ii) an inner shaft within the lumen of the outer sheath, wherein the inner shaft has a lumen; and (b) a valve prosthesis comprising a frame structure comprising one or more anchor retaining elements and one or more anchors configured to be disposed within the one or more anchor retaining elements in a delivery configuration and at least partially exposed from the one or more anchor retaining elements in a deployed configuration, wherein the one or more anchors are configured to be advanced out of the one or more anchor retaining elements into the deployed configuration when the frame structure is in an expanded configuration, and wherein the frame structure is maintained in an unexpanded configuration by radial constriction from the outer sheath.

The delivery device can further comprise a guidewire disposed within a lumen of the inner shaft. The one or more anchors can comprise a spiral shape in the deployed configuration. The one or more anchors can comprise an elongated shape when disposed within the one or more anchor retaining elements. The one or more anchors can comprise one or more locking mechanisms configured to maintain the anchor in the deployed configuration. The one or more locking mechanisms can comprise a frictional band, a polymer coating, or one or more key and one or more key hole features.

The one or more anchors can comprise a super-elastic material. The one or more anchors can comprise nitinol. The frame structure can comprise an expandable stent. The expanded configuration can be a generally tubular expanded shape. The frame structure can comprise an expanded outer periphery in the expanded configuration and a compressed outer periphery when subject to an external radial force in the unexpanded configuration, wherein the compressed outer periphery is smaller in diameter than the expanded outer periphery. The frame structure can be balloon-expandable. The frame structure can be self-expanding. The free end can comprise an atraumatic tip. The free end can comprise a ball tip. The free end can be configured for piercing tissue.

The one or more anchors can comprise a spiral band. The spiral band can comprise a spiral wire. The spiral band can comprise a planar spiral band. The spiral band can comprise at least one channel or lumen disposed therein. The spiral band can comprise a hollow spiral band. The at least one channel or lumen can comprise a stiffening member disposed therein. The spiral band can have a circular, tubular, hollow, square, elongated, or triangular cross-section. The spiral band can comprise a tapered spiral band. The tapered spiral band can be configured to taper from a first end of the tapered spiral band to the free end. The first end can be a proximal end and the free end can be a distal end.

The frame structure can be configured for expanding within the native valve of the patient. The unexpanded configuration can be sized and dimensioned for percutaneous insertion and the expanded configuration is sized and dimensioned for implantation in the native valve of the patient. The frame structure can comprise a first and second opposite ends, the first end extending above a native valve and the second end extending below the native valve when the frame structure is anchored to the native valve. The frame structure can sit below the native valve when the frame structure is anchored to the native valve.

The system can further comprise a valve segment within the frame structure comprising a biocompatible one-way valve. At least a portion of the valve segment can be positioned within at least a portion of the frame structure. The valve segment can comprise at least one leaflet having an inner layer and an outer layer, and wherein the frame structure is attached to the outer layer at one or more ends of the frame structure. The valve segment can comprise a plurality of leaflets.

Disclosed herein is a method for treating a diseased native valve in a patient, the method comprising (a) advancing an outer sheath to a native valve; (b) advancing a valve prosthesis from a lumen of the outer sheath; (c) expanding a frame structure of the valve prosthesis within the native valve from an unexpanded configuration to an expanded configuration; (d) advancing one or more anchors from one or more anchor retaining elements disposed along a lateral side of the frame structure; (e) securing the one or more anchors to one or more structures of the native valve; (f) retracting the outer sheath from the native valve. Advancing the one or more anchors from the one or more anchor retaining elements can be actuated by one or more proximal anchor pushing element attached to one or more anchors via one or more anchor releasing elements. Expanding the frame structure of the valve prosthesis within the native valve from an unexpanded configuration to an expanded configuration, (c), can further comprise one or more actuation arms to release the valve prosthesis from constraint, wherein the valve prosthesis is maintained within the outer sheath by radial constriction. The outer sheath can be steerable.

The one or more anchors can be a spiral band. The spiral band can comprise a free end and wherein anchoring comprises wrapping the free end of the anchor around one or more structures of the native valve. The one or more structures can comprise one or more native chordae tendineae of native valve. The valve prosthesis can comprise a valve segment therewithin comprising a biocompatible one-way valve. The native valve can be in a heart of a patient.

The method can further comprise transseptally inserting the distal end of the outer sheath into a left atrium of the heart. The native valve can comprise a mitral valve, wherein a first side of the native valve comprises a left atrium, and wherein a second side of the native valve comprises a left ventricle. The outer sheath can be advanced from the first side of the native valve. Securing the one or more anchors to one or more structures of the native valve can comprise retracting one or more proximal anchor pusher elements releasably connected to the one or more anchors via one or more anchor releasing elements and releasing the one or more anchors from the one or more proximal anchor pusher elements, wherein the one or more proximal anchor pusher elements are threaded through a lumen of the inner shaft. Advancing the one or more anchors from the one or more anchor retaining elements can comprise advancing the one or more proximal anchor pusher elements releasably attached to the one or more anchors through the lumen of the inner shaft.

Disclosed herein is a valve prosthesis, comprising: a frame having an annular shape for placement within a diseased native valve; and one or more anchor elements slidably engaged with the frame, the one or more anchor elements configured to extend distally and radially outward from the frame such that a distal end of the one or more anchor elements takes on a curved shape for wrapping around one or more structures of the diseased native valve, thereby securing the frame to the diseased native valve. The distal end of the one or more anchor elements can be configured to extend proximally with respect frame when extended from the frame. The frame can be configured to transition between a compressed configuration and an expanded configuration. The frame can be configured to transition from the compressed configuration to the expanded configuration upon retraction of an outer sheath from the frame or distal advancement of the frame with respect to the outer sheath. The frame can include a mesh. The frame can include a stent. The frame can include a metal material. The frame can include a flexible tube. At least a portion of the one or more anchor elements can include a wire or band. The curved shape of the one or more anchor elements may include a hook shape. At least a portion of the one or more anchor elements can be made of a shape memory material. The frame can include one or more anchor retaining elements configured to retain the one or more anchor elements to the frame. The one or more anchor retaining elements can be arranged vertically along a lateral side of the frame. The one or more anchor elements can include an atraumatic tip. Once the one or more anchor elements are deployed from the frame, the one or more anchor elements can be configured be pulled in a proximal direction to a locked position by one or more anchor retaining elements. The frame can include one or more prosthetic valve leaflets. The valve prosthesis can be configured to transition between a delivery configuration to a deployed configuration. The one or more anchor elements can have a straight shape when in the delivery configuration and the curved shape when in the deployed configuration. The frame can be in a radially reduced state when in the delivery configuration and a radially expanded state when in the deployed configuration. The valve prosthesis can be configured to be in contact with one or more actuation arms while the frame is in a compressed configuration within an outer sheath, wherein the one or more actuation arms is configured to push the valve prosthesis out the outer sheath.

Disclosed herein is a method of treating a diseased valve, comprising: advancing a valve prosthesis in a delivery configuration within the diseased valve, wherein the valve prosthesis includes one or more anchor elements coupled to a frame, the frame and the one or more anchor elements being in a radially reduced state when in the delivery configuration; expanding the frame to a radially expanded state within the diseased valve; and deploying the one or more anchor elements by distally and radially extend the one or more anchor elements with respect to the frame, wherein, during deployment, the one or more anchor elements take on a curved shape and wrap around one or more structures of the diseased native valve, thereby securing the valve prosthesis to the diseased native valve. The one or more anchor elements can be deployed after the frame is expanded. The one or more anchor elements can be deployed while the frame is expanded. Deploying the one or more anchor elements can comprise sliding the one or more anchor elements distally with respect to the frame. Deploying the one or more anchor elements can comprise pushing one or more anchor pushing elements that are proximally coupled to the one or more anchor elements. The method can further comprise locking the one or more anchors in place by pulling or rotating the one or more anchors once deployed. The one or more anchor elements can take on hook shape where distal ends of the one or more anchor elements point in a proximal direction once deployed. The one or more anchor elements can have a straight shape when in the delivery configuration.

These and other aspects and embodiments are described in detail in the following description related to the appended drawing figures.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings of which:

FIGS. 1A-1H show delivery of a frame structure to a native heart valve wherein anchor elements are delivered simultaneously with or after the frame structure wherein the anchor elements loop around the chordae tendineae where the chordae tendineae attach to the leaflets of the heart valve, in accordance with embodiments.

FIGS. 2A-2B show delivery of another embodiment of a frame structure to a native heart valve wherein the anchor elements are delivered simultaneously with or after the frame structure wherein the anchor elements spiral around the chordae tendineae, in accordance with embodiments.

FIG. 3 shows a top view of a frame structure comprising a valve prosthesis and an anchor delivery retaining elements, in accordance with embodiments.

FIGS. 4A-4D show delivery steps of the frame structure of FIGS. 2A-2B as the anchor elements are pushed through the anchor delivery retaining elements of the frame structure by proximal anchor pushing elements of the delivery device into their delivery configuration, at which point the anchor releasing element can be detached from the anchor delivery elements, in accordance with embodiments.

FIG. 5 shows a side cross section of the inner delivery shaft of a delivery device, and the valve prosthesis in a delivery configuration within the outer sheath of the delivery device, in accordance with embodiments.

FIGS. 6A-6B show cross sections A-A and B-B, respectively, of the system of FIG. 5, in accordance with embodiments.

### DETAILED DESCRIPTION

In the following detailed description, reference is made to the accompanying figures, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Although certain embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments, however, the order of description should not be construed to imply that these operations are order dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

The present disclosure is described in relation to deployment of systems, devices, or methods for treatment of a diseased native valve of the heart, for example a mitral valve. However, one of skill in the art will appreciate that this is not intended to be limiting and the devices and methods disclosed herein may be used in other anatomical areas and in other surgical procedures.

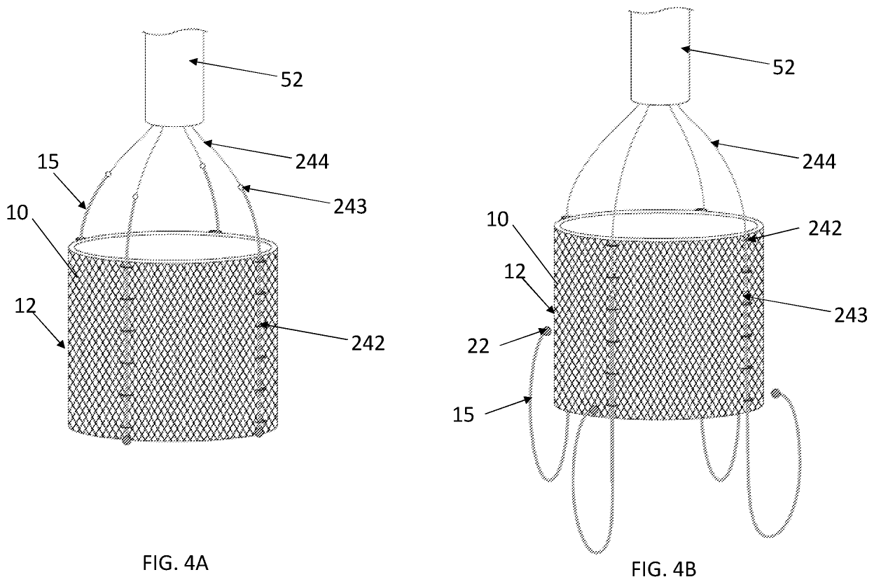

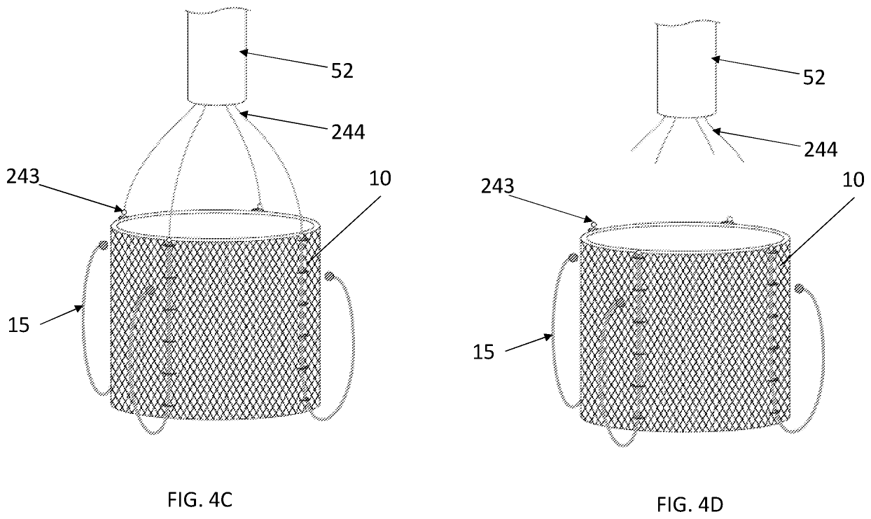

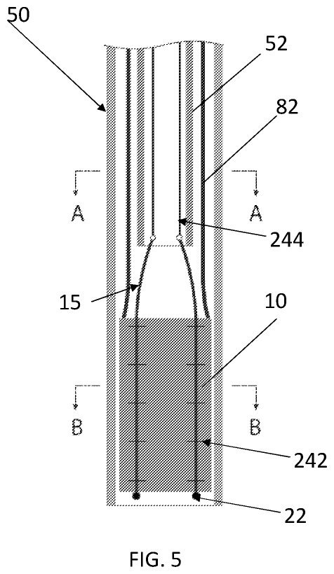

The valve prostheses described herein may include an annular frame (e.g., frame structure 12, FIGS. 4A-4D), retaining elements (e.g., retaining elements 242, FIGS. 4A-4D), and one or more anchors (e.g., anchors 15, FIGS. 4A-4D). The anchors (also referred to as “anchor elements”) may be configured to have a stowed configuration (e.g., delivery configuration) and a deployed configuration. When deployed, the one or more anchors may be configured to engage with one or more anatomical structures of a native valve, or near the native valve. In some examples, the one or more anchors may include one or more wires or bands. The one or more anchors may have a variety of shapes as understood herein. For example, an anchor may be shaped to include one or more loops, curves, bends, twists and/or corners. In some cases, an anchor may include one or more protrusions and/or indentations, such as one or more barbs, hooks, prongs and/or spikes. The retaining elements may be configured to (e.g., slidably) retain the one or more anchors to the frame. In some examples, the retaining elements include one or more eyelets or tubes on or within the frame. When deployed, the one or more anchors may extend distally and radially outward with respect to the frame. In some cases, the one or more anchors may curve back to point proximally and/or in an atrial direction when deployed.

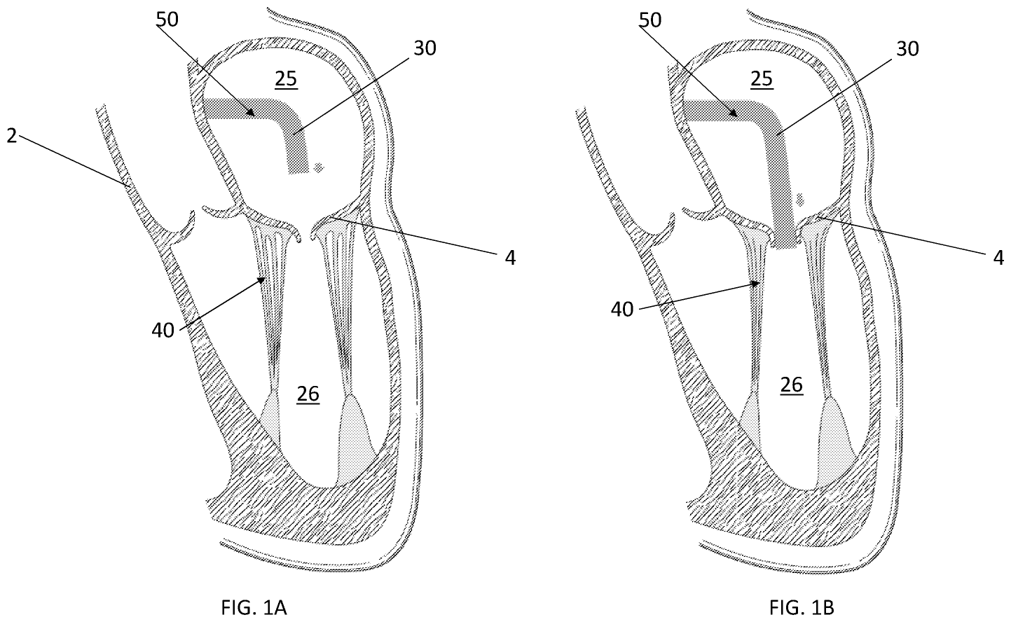

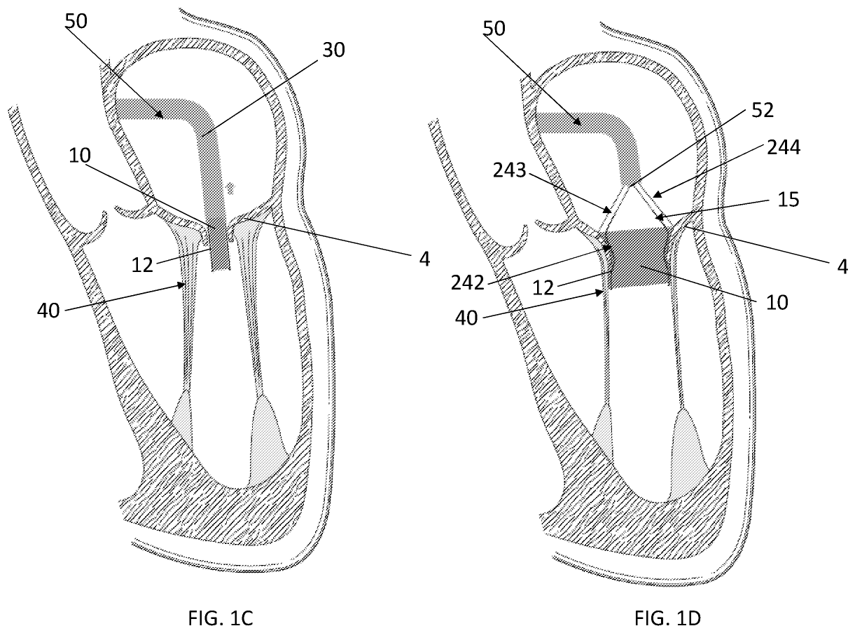

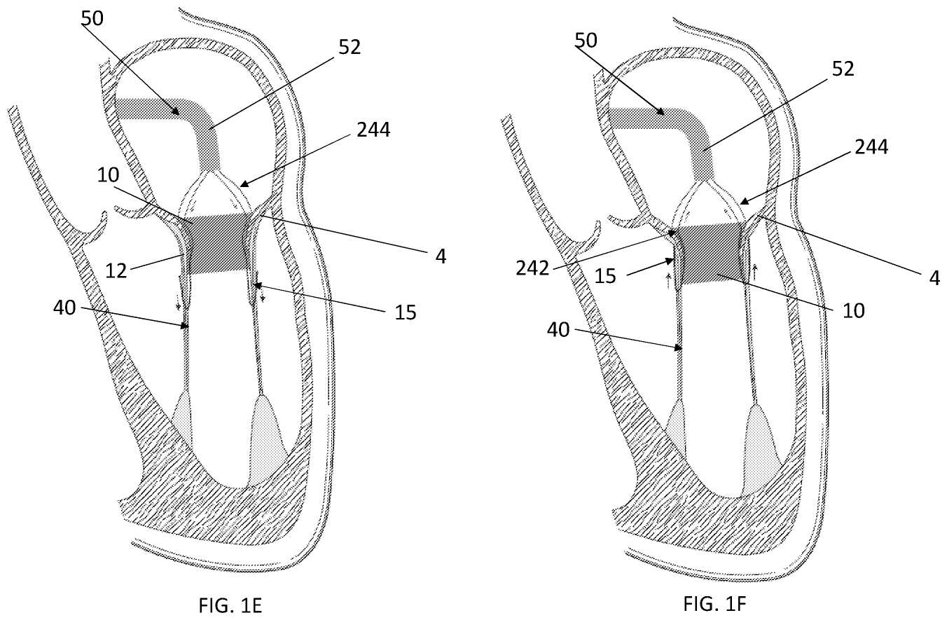

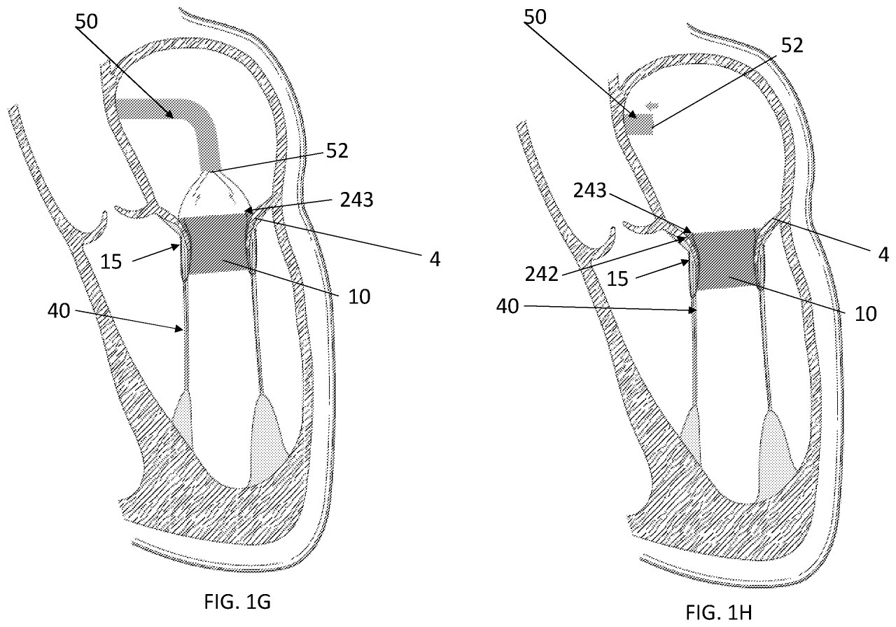

FIG. 1A is a section view of a delivery device 30 being guided into a left atrium 25 of a heart 2. The delivery device 30 may be guided over a guide wire as will be understood by one of ordinary skill in the art based on the teachings herein. The delivery device 30 may comprise an outer sheath 50. The outer sheath 50 may contain an inner shaft 52 which is separate from the valve prosthesis 10. The distal end of the outer sheath 50 may be relatively rigid such that it can exert a compression force on a frame structure 12 delivered within the outer sheath 50, while still retaining flexibility to deform under an external force. The outer sheath 50 may comprise a flexible material. The inner shaft 52 may be located in a lumen of the outer sheath 50. The delivery device 30 can be steered by movement of the outer sheath 50 or alternatively a guidewire. The outer sheath 50 can be steered by an advancement of a throwdown arm through the lumen of the outer sheath 50 as described herein.

FIG. 1B is a section view of the delivery device 30 being advanced from the left atrium 25 into the left ventricle 26 through the native mitral valve 4. As the delivery device 30 is advanced further into the left atrium 25, the distal end of the outer sheath 50 may be placed through the valve 4 of the heart 2. The inner shaft 52 may comprise a tight bend configured align the inner shaft 52 and/or distal tip of the outer sheath 50 with the mitral valve 4. Correct alignment of the distal tip with the mitral valve 4 may facilitate deployment of the valve prosthesis 10 into the valve 4 and the deployment of the anchor 15 around the chordae tendineae 40. In some embodiments, the distal end of the outer sheath may be steerable. The distal end of the outer sheath may be steered such that the distal end points towards the native valve 4.

FIG. 1C is a section view of the delivery device 30 comprising the outer sheath 50 and a valve prosthesis 10 in an unexpanded configuration. The valve prosthesis 10 may comprise a frame structure 12 and one or more anchors 15 as described herein. The valve prosthesis 10 may be held in the distal end of the outer sheath 50 or advanced into the distal end of the outer sheath 50 after the outer sheath 50 is positioned adjacent the native valve 4 (e.g., by an inner shaft as described herein). Upon positioning the distal end of the outer sheath 50 within the valve 4, the outer sheath 50 may be retracted relative to the valve prosthesis 10.

FIG. 1D is a section view of the delivery device 30 comprising the outer sheath 50, the valve prosthesis 10, and the inner shaft 52. Upon retraction of the outer sheath 50, as can be seen in FIG. 1C, the valve prosthesis 10 may be released from the outer sheath 50 (e.g., frame structure 12 may be released from radial constriction of the outer sheath 50 as described herein) into an expanded configuration within the native valve 4. The distal end of the outer sheath 50 can be retracted to be aligned with the distal end of the inner shaft 52. The retraction of the outer sheath 50 can expose the proximal anchor pushing elements 244. The one or more proximal anchor pushing elements 244 may be coupled to one or more anchor releasing elements 243.

One or more anchors 15 can be located in a delivery configuration within one or more anchor delivery retaining elements 242 located vertically along a lateral side of the frame structure 12.

FIG. 1E is a section view of the delivery device 30 comprising the outer sheath 50, the inner shaft 52, and the proximal anchor pushing element 244. After the frame structure 12 has been expanded, the one or more proximal anchor pushing elements 244 can be pushed distally, toward the native valve 4, to release the one or more anchors 15 from a delivery configuration within the anchor delivery retaining elements 242 to a deployed configuration at least partially exposed from the delivery retaining elements 242. The one or more anchors 15 may be configured to wrap proximally and around the chordae tendineae 40 and/or leaflets of the native valve 4. The one or more anchors 15 may extend distally and radially outward from the frame structure 12. The one or more anchors 15 may be configured to take on a curved shape when extended from the frame structure 12.

Note that in some cases, expansion of the frame structure 12 (e.g., FIG. 1D) and release of the one or more anchors 15 (e.g., FIG. 1E) may occur in one process (e.g., simultaneously).

FIG. 1F is a section view of the delivery device 30 comprising the outer sheath 50, the inner shaft 52, and the proximal anchor pushing elements 244. The one or more proximal anchor pushing elements 244 can be retracted (e.g., pulled) toward the outer shaft 50, effectively pulling the anchor(s) 15 into a locked position around the chordae tendineae 40 to lock the valve prosthesis 10 into position within the native valve 4, in some embodiments the anchor releasing elements 243 are at a proximal end of the valve prosthesis 10. In some cases, the one or more proximal anchor pushing elements may be rotated (e.g., instead of or in addition to pulling) to place the anchor(s) 15 in a locked position.

In some embodiments, the valve prosthesis 10 can be locked into place by at least 10, 9, 8, 7, 6, 5, 4, 3, 2, or 1 anchor element 15. Each anchor 15 can be held in a delivery configuration against the valve prosthesis 10 by at least 1, 2, 3, 4, 5, 6, 7, 8, 9, 10, 11, 12, 13, 14, 15, 16, 17, 18, 19 or 20 anchor retaining elements 242. In some cases, the anchor retaining elements 242 can be molded as part of the frame structure 12. The anchor retaining elements 242 can be secured to the frame structure 12. The anchor retaining elements 242 can be configured to create low friction with the anchor 15. Alternatively, the anchor retaining elements 242 can be configured to create high friction with the anchor 15.

FIG. 1G is a section view of the delivery device 30 comprising the outer sheath 50, the inner shaft 52, and the proximal anchor pushing elements 244. The one or more anchor releasing elements 243 can be locked into the one or more anchor retaining elements 242 prior to release from the one or more proximal anchor pushing elements 244. The one or more proximal anchor pushing elements 244 can be released from the one or more anchors 15 at one or more anchor releasing elements 243.

FIG. 1H is a section view of the delivered configuration of the valve prosthesis 10 within the native valve 4 and the removal of the delivery device 30. In some embodiments, the proximal anchor pushing elements 244 may be withdrawn into the inner sheath 52 during the removal of the delivery device 30 from the left atrium 25.

Although the steps above show a method of delivering a valve prosthesis in accordance with embodiments, a person of ordinary skill in the art will recognize many variations based on the teaching described herein. The steps may be completed in a different order. Steps may be added or deleted. Some of the steps may comprise sub-steps. Many of the steps may be repeated as often as necessary ensure correct placement of the delivery device and deployment of the valve prosthesis components.

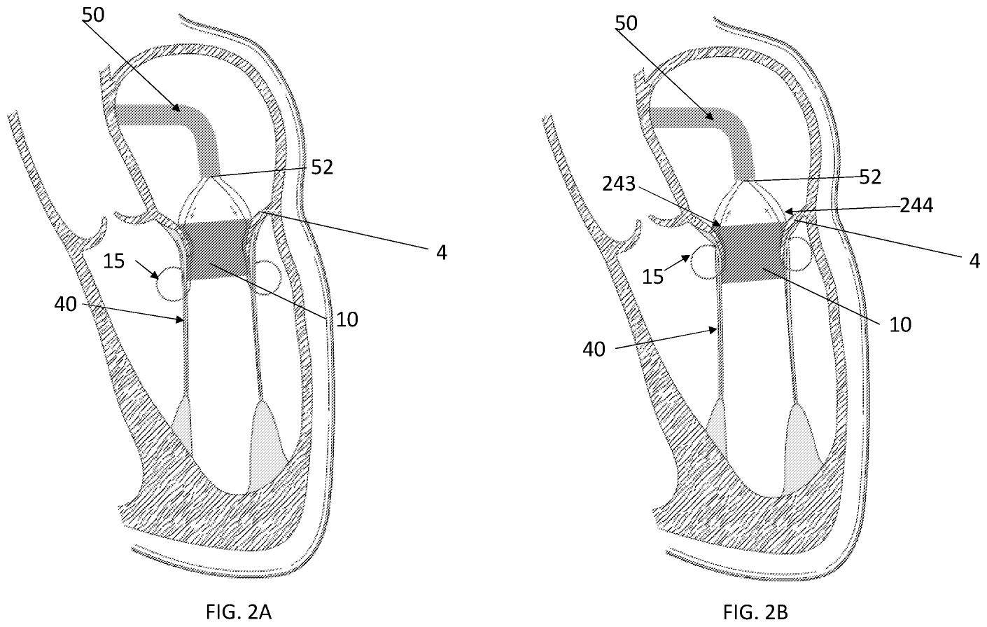

FIG. 2A shows another embodiment of a valve prosthesis 10 having one or more anchors 15 configured to be deployed following deployment of the frame structure 12. The anchors 15 may be substantially similar to the anchors 15 of FIGS. 1C-1H except they may be more curved or rounded shape (e.g., larger radius of curvature) compared to the anchors 15 of FIGS. 1C-1H. In some embodiments, one or more anchor 15 may comprise a spiral band or a flat band. FIG. 2A is a section view of the delivery device 30 comprising the outer sheath 50, the inner shaft 52, and the proximal anchor pushing element 244. The one or more proximal anchor pushing elements 244 can be pushed toward the native valve 4 to release the one or more anchor 15 from a delivery configuration within the anchor delivery retaining elements 242 to a deployed configuration around the chordae tendineae 40.

FIG. 2B is a section view of retraction of the delivery device 30 comprising the outer sheath 50, the inner shaft 52, and the proximal anchor pushing elements 244. The one or more proximal anchor pushing elements 244 can be retracted toward the outer shaft 50, effectively pulling the anchor into a locked position around the chordae tendineae 40 to lock the valve prosthesis into position within the native valve 4. The one or more anchors may have a portion with a large radius of curvature to minimize friction when the anchor is in the delivery system as compared to the one or more anchors as seen in FIGS. 1E-1H.

The valve prosthesis 10 may be operably coupled to the delivery device 30 by proximal anchor pushing elements 244, as described herein. In some embodiments, at least a portion of the valve prosthesis 10 may be directly coupled to the inner shaft 52. Alternatively, or in combination, at least a portion of the valve prosthesis 10 may be indirectly coupled to the inner shaft 52.

The outer sheath 50 may be configured to deliver an anchor 15 of the valve prosthesis 10 to the native valve 4. The outer shaft 50 can form a tight bend (e.g., a 90 degree angle), thereby centering the distal end of the delivery device 30 to the center of the mitral valve 4. The inner sheath 52, coupled to proximal anchor pushing elements 244, can then push the anchor 15 through the anchor delivery retaining elements and around the chordae tendineae 40. The proximal anchor retaining elements 243 may be configured to correctly orient the anchor 15 relative to the bent inner shaft 52 in order to facilitate wrapping of the anchor 15 around the chordae tendineae 40 as the anchor 15 is deployed from the delivery configuration to the deployed configuration.

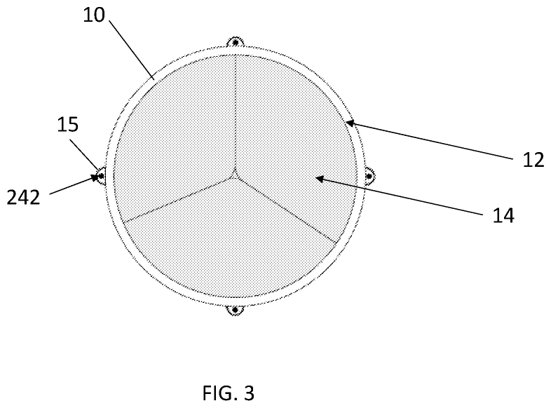

FIG. 3 is a top view of a valve prosthesis 10. The valve prosthesis 10 may comprise a frame structure 12 and one or more anchors 15 (four anchors 15 are shown) disposed within one or more anchor retaining elements 242 (four anchor retaining elements are shown) coupled to the frame structure 12. The anchor retaining elements 242 may be molded as a part of the frame structure 12. The anchor retaining elements 242 may be fastened to the frame structure 12. In some cases, the anchor retaining elements 242 may comprise a shape memory alloy (e.g., Nitinol (NiTi)), stainless steel, or Titanium (Ti).

The anchor retaining elements can be configured to guide the positioning of the anchor 15 to the chordae tendineae 40. The anchor retaining elements 242 may be positioned in sequence along a lateral edge of the frame structure 12. The anchor retaining elements 242 may be positioned in a diagonal line from the proximal end of the frame structure to the distal end of the frame structure. The anchor retaining elements 242 may be positioned in a straight line from the proximal end of the frame structure to the distal end of the frame structure.

The anchor retaining elements 242 can be configured to lock the anchor 15 into place after positioning around the chordae tendinea 40. The anchor retaining elements 242 can be configured to reduce friction with the anchor 15 when the anchor 15 is pushed toward the chordae tendinea 40. The anchor retaining elements 242 can be configured to increase friction with the anchor 15 when the anchor 15 is pulled up toward the inner shaft 52. The anchor retaining elements 242 can be configured to lock the anchor releasing element 243 when the anchor releasing element 243 is released from the proximal anchor pushing element 244. The one or more anchors 15 can be locked into place by a latch.

The valve prosthesis 10 may comprise a valve segment 14 disposed therein. Valve segment is used somewhat interchangeably with prosthetic valve leaflet and generally refers to the prosthetic leaflets and frame. As used herein, “prosthetic valve” may refer to all manner of prosthetic and artificial replacement valves including tissue (biological valves), tissue-engineered valves, polymer valves (e.g., biodegradable polymer valves), and even certain mechanical valves. The valve segment can be similar to existing transcatheter valves. The valve segment can be similar to existing surgical tissue valves, and mechanical valves. At least a portion of the valve segment may be positioned within at least a portion of the valve prosthesis 10, for example with a frame structure of the valve prosthesis. The valve segment may include leaflets formed of multi-layered materials for preferential function. The valve segment may comprise at least one leaflet having an inner layer and an outer layer. The valve segment may be attached to a valve structure which is in turn connected to the valve prosthesis 10. The valve structure may be connected to the valve prosthesis 10 before or after the valve prosthesis 10 has been deployed adjacent a native valve. The valve segment may be attached directly to the valve prosthesis 10. The valve prosthesis 10 may be attached to a leaflet, for example an outer layer of a leaflet, at one or more ends of the valve prosthesis 10. The valve prosthesis 10 may be attached to a leaflet, for example an outer layer of a leaflet, at one or more intermediate portions of the valve prosthesis 10. The valve segment may comprise a plurality of leaflets. The valve segment may comprise a biocompatible one-way valve. Flow in one direction may cause the leaflet(s) to deflect open and flow in the opposite direction may cause the leaflet(s) to close.

FIG. 4A shows a side view of the valve prosthesis 10 after expansion of the frame structure 12 but before advancement of the anchor 15 from the delivery configuration within the anchor retaining elements 242 to a deployed configuration. The frame structure 12 may be made of any of a variety of materials. The frame structure 12 may include one or more rigid materials and/or one or more flexible materials. In some cases, the frame structure 12 may include an expandable mesh that is made of metal and/or polymer material. The mesh may have a regular or irregular arrangement (e.g., pattern) of structural elements (e.g., wires, bars and/or struts). In some cases, the frame structure 12 may include a flexible tube made of a flexible material (e.g., flexible polymer material). The frame structure 12 may include both a mesh (e.g., metal and/or polymer stent) and a flexible tube.

The anchor retaining element 242 may have any of a variety of shapes. In some cases, an anchor retaining element 242 may an annular shape with one or more openings configured to receive a corresponding anchor 15. For example, an anchor retaining element 242 may include a grommet (e.g., eyelet) and/or a tube. In some cases, the anchor retaining elements 242 may be positioned on a side (e.g., inside and/or outside) of the frame structure 12. In some cases, the anchor retaining elements 242 may be positioned within the frame structure. In some instances, the anchor retaining elements 242 may be made of the same material(s) as the frame structure 12. In some instances, the anchor retaining elements 242 may be made of a different material than the frame structure 12.

The proximal anchor pushing elements 244 can be located at least partially within a lumen of the inner shaft 52. One or more proximal anchor pushing elements 244 can be coupled to one or more anchors 15 via one or more anchor releasing elements 243. Each anchor 15 can be threaded through an anchor delivery retaining element 242 located (e.g., vertically) along a side of (and/or within) the frame structure 12 of the valve prosthesis 10.

FIG. 4B shows a side view of the valve prosthesis 10 after advancement of the one or more anchors 15 distally out of the anchor retaining elements 242. One or more anchor releasing elements 243 can be pushed through the anchor delivery retaining elements 242. One or more anchors 15 can be pushed through the anchor delivery retaining elements 242 and released to capture the chordae tendineae between the anchor 15 and the frame structure 12. Upon release from the anchor delivery retaining elements 242, the anchor 15 may form one or more loops. The free end 22 of each anchor 15 may comprise a ball tip or other atraumatic tip as will be understood by one of ordinary skill in the art. The free end 22 of the anchor 15 may be configured to sit proximal to the distal end of the frame structure 12 such that the anchor 15 effectively reverses direction as it is deployed distally from the anchor retaining elements 242.

The one or more anchors 15 may have any of a variety of shapes. In some cases, at least a portion of the one or more anchors 15 may be shaped to include one or more loops, curves, bends, twists, corners, zigzags and/or other shapes. In some cases, at least a portion of an anchor 15 may include one or more surface features, such as one or more protrusions and/or indentations. Such surface features may improve engagement with, for example, tissue. For instance, an anchor may include one or more barbs, hooks, prongs, spikes and/or divots.

FIG. 4C shows a side view of the valve prosthesis 10 in a locked configuration. The valve prosthesis 10 may be placed into a locked configuration by pulling the one or more anchor releasing elements 243 to an anchor retaining element 242 that is at the end of the valve prosthesis 10 that is closest to the inner shaft 52. The anchor body 15 and free end 22 may be adjusted proximally in order to bring the anchors 15 closer to the native valve.

FIG. 4D shows a side view of the valve prosthesis 10 after the release of one or more anchor releasing elements 243 from one or more proximal anchor pushing elements 242.

The anchor 15 may form one or more loops. In some embodiments, a loop may comprise a shape that bends back towards its origin but does not cross itself, making a rotation within a range of about 180 degrees to about 360 degrees. For example, a loop may comprise an arc having a central angle within a range of about 180 degrees to about 360 degrees. In at least some embodiments, the one or more loops may comprise an arc. In some embodiments, a loop may comprise a shape that bends back towards and crosses itself, making at least a 360 degree rotation. In at least some embodiments, the one or more loops may comprise a 360 degree rotation (for example, a circle). In some embodiments, the one or more loops may comprise a 360 degree to 720 degree rotation (for example, a loop crossing itself once and rotating further towards a second crossing and formation of a second 360 loop).

The one or more loops may comprise any number of loops desired, for example, one, two, three, four, five, six, seven, eight, nine, or ten loops. The one or more loops may comprise a rotation within a range of about 180 degrees to about 3600 degrees. The one or more loops may comprise a rotation within a range bounded by any of the following values: 180 degrees, 270 degrees, 360 degrees, 450 degrees, 540 degrees, 630 degrees, 720 degrees, 810 degrees, 900 degrees, 990 degrees, 1080 degrees, 1170 degrees, 1260 degrees, 1350 degrees, 1440 degrees, 1530 degrees, 1620 degrees, 1710 degrees, 1800 degrees, 1890 degrees, 1980 degrees, 2070 degrees, 2160 degrees, 2250 degrees, 2340 degrees, 2430 degrees, 2520 degrees, 2610 degrees, 2700 degrees, 2790 degrees, 2880 degrees, 2970 degrees, 3060 degrees, 3150 degrees, 3240 degrees, 3330 degrees, 3420 degrees, 3510 degrees, or 3600 degrees.

FIG. 5 shows a section view of the delivery configuration of the valve prosthesis 10 within an outer sheath 50. One or more actuation arms 82 may be positioned in contact with the valve prosthesis 10 so as to push the valve prosthesis 10 away from the inner shaft 52 and out of the outer sheath 30. The proximal anchor pushing elements 244 can be threaded through a lumen of the inner shaft 52. The anchor retaining elements 242 can be positioned vertically along the length of the frame structure 12. The anchor 15 can comprise a ball tip or other atraumatic tip at its free end 22.

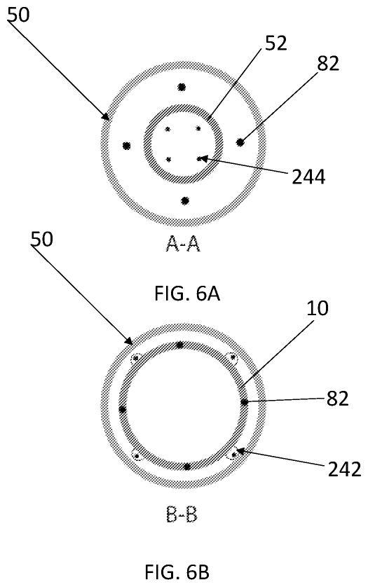

FIG. 6A shows an A-A section view of the configuration of FIG. 5 wherein one or more actuation arms 82 are threaded through a lumen of the outer sheath 50 and outside of an inner shaft 52. The proximal anchor pushing elements can be threaded through a lumen of the inner shaft 52.

FIG. 6B shows a B-B section view of the configuration of FIG. 5 wherein the valve prosthesis 10 is maintained in a delivery configuration by radial constriction from the outer sheath 50. In some embodiments, one or more actuation arms 82 can be threaded through one or more lumens of the outer edge of the valve prosthesis 10. One or more anchor retaining elements 242 can be positioned on an outside edge of the valve prosthesis.

The valve prosthesis may be substantially similar to any of the valve prostheses described in U.S. patent applications Ser. No. 16/546,901, 16/594,946, 16/723,537, and International Patent Applications No. PCT/US2019/047542, PCT/US2019/055049, PCT/US2019/057082, PCT/US2019/068088, PCT/US2020/023671, PCT/US2020/027744, and PCT/US2020/058413, which are incorporated herein by reference in their entireties for all purposes.

The valve prosthesis 10 may be delivered to a heart of a patient with a delivery device as described herein. For example, the delivery device may comprise an outer sheath assembly (e.g., an outer catheter), an inner shaft (e.g., a delivery tube) disposed within a lumen of the outer sheath, and an optional guidewire disposed within a lumen of the inner shaft. The delivery device may comprise an outer sheath assembly comprising an outer sheath and a distal end of the outer sheath assembly. As described herein, an inner shaft may be disposed within a lumen of the outer sheath assembly and a guidewire may be disposed adjacent to the inner shaft within a lumen of the outer sheath assembly. Alternatively, the guidewire may be disposed in a lumen of inner shaft. The guidewire may optionally comprise a nosecone to facilitate guidance of the guidewire to the native valve. A proximal end of the valve prosthesis 10 may be operably coupled to the inner shaft during delivery to the native valve as described herein. The outer sheath may be steerable.

The valve prosthesis 10 may be operably coupled to the delivery device 30 as described herein. In some embodiments, at least a portion of the valve prosthesis 10 may be directly coupled to the inner shaft 52. Alternatively, or in combination, at least a portion of the valve prosthesis 10 may be indirectly coupled to the inner shaft 52.

Any of the anchors 15 described herein may comprise a delivery (e.g., elongated, radially collapsed, or unexpanded) configuration and a deployed configuration. A frame structure of the valve prosthesis (e.g., as can be seen in FIG. 4A) may remain in its expanded configuration while the anchor 15 is in the delivery configuration. In various embodiments, the anchor may be self-expanding and may move to the deployed configuration as it is removed from the anchor delivery retaining elements 242. In various embodiments, the anchor 15 may be configured to self-assemble when it is deployed in the heart cavity (e.g., left ventricle or left atrium). The anchor 15 may be actuated from the delivery configuration to the deployed configuration adjacent the native valve using any method or mechanism understood by one of ordinary skill in the art from the description herein.

Additional description for the delivery device and other similar delivery devices usable in the embodiments described herein may be found in U.S. patent applications Ser. No. 16/546,901, 16/594,946, 16/723,537, and International Patent Applications No. PCT/US2019/047542, PCT/US2019/055049, PCT/US2019/057082, PCT/US2019/068088, PCT/US2020/023671, PCT/US2020/027744, and PCT/US2020/058413, which are incorporated herein by reference in their entireties for all purposes.

The anchor 15 may comprise a band or wire 20 having a free end 22. The other end of the anchor 15 may be coupled to the top (proximal end) or bottom (distal end) of a frame structure (e.g., frame structure 12 shown in FIG. 4A) as described herein. Alternatively, or in combination, the other end of the anchor 15 may not be attached to a frame structure as described herein. The anchor 15 may be configured to wrap at least partially around a frame structure in the deployed configuration.

The anchor 15 may be fully advanced from a first side of a native valve in a patient (e.g., an atrial side) to a second side of the native valve (e.g., into a ventricle of the heart) and anchor a frame structure to the native valve when the frame structure is in the expanded configuration adjacent the native valve.

The anchor 15 may, for example, be wrapped around the chordae tendineae 40 by further advancing the anchor 15 through the anchor retaining elements 242. The anchor 15 may be wrapped around the native chordae tendineae 40 by being pushed out of the anchor delivery retaining elements 242 positioned radially on an outside edge of the valve prosthesis 10. The anchor retaining elements 242 may direct the anchor to a direction transverse to the longitudinal axis of the outer sheath 50 while centered about the longitudinal axis of the outer sheath 50.

The anchor 15 may be wrapped upwards around the native chordae tendineae 40 in order to lock the valve prosthesis 10 in place as described herein. The frame structure 10 may be released from the delivery device 30 as described herein. The delivery device 30 may then be retracted to leave the valve prosthesis 10 in place.

As described herein, the valve prosthesis 10 may comprise a frame structure 12 and an anchor 15. The anchor 15 may be directly coupled to the frame structure 12, for example at a proximal or distal end thereof or along a lateral side thereof. Alternatively, or in combination, the anchor 15 may be detachably and/or slidably coupled to the delivery device 30 prior to deployment at the native valve. For example, a proximal end of the anchor 15 may be detachably coupled to the inner shaft 52 during delivery to the native valve. Alternatively, or in combination, a proximal end of the anchor 15 may be coupled to a distal end of the frame structure 12 or a proximal end of the frame structure 12. The frame structure 12 may have an unexpanded configuration, for example when the valve prosthesis 10 is in its unexpanded configuration, and an expanded configuration, for example when the valve prosthesis 10 is in its expanded configuration. The expanded configuration may have a generally tubular expanded shape. The frame structure 12 may be expanded within the native valve of the patient. In some embodiments, the unexpanded configuration may be sized and dimensioned for percutaneous insertion and the expanded configuration may be sized and dimensioned for implantation in the native valve of the patient.

The frame structure 12 may comprise a first and second opposite ends, the first end extending above a native valve and the second end extending below the native valve when the frame structure 12m is anchored to the native valve. Alternatively, the frame structure 12 may be configured to sit entirely below the native valve when the frame structure 12 is anchored to the native valve.

In some embodiments, the frame structure 12 may comprise an expanded outer periphery in the expanded configuration and a compressed outer periphery when subject to an external radial force in the unexpanded configuration. The compressed outer periphery may be smaller in diameter than the expanded outer periphery.

The frame structure 12 may be balloon-expandable, self-expanding, or otherwise expansible as will be understood by one of ordinary skill in the art. The frame structure 12 may, for example, comprise an expandable stent.

The delivery system 30 may comprise an inflatable balloon disposed within the frame structure 12 and inflation of the balloon may cause expansion of the frame structure 12 as described herein.

Alternatively, or in combination, the frame structure 12 may be self-expanding and may be maintained in the unexpanded configuration by radial constriction from the outer sheath 50 of the delivery device 30. Advancement of the inner shaft 52 out of the lumen of the outer sheath 50 may actuate the frame structure 12 into the expanded configuration. Advancement of the inner shaft 52 and/or one or more actuation arms 82 out of the lumen of the outer sheath 50 may actuate the frame structure 12 into the expanded configuration.

The frame structure 12 and/or anchor 15 may be operably coupled to the delivery device 30 as described herein. In some embodiments, at least a portion of the frame structure 12 and/or anchor 15 may be directly coupled to the inner shaft 52. For example, a proximal portion of the frame structure 12 and/or a proximal portion of the anchor 15 may be coupled to a distal portion of the inner shaft 52. Alternatively, or in combination, at least a portion of the frame structure 12 and/or anchor 15 may be indirectly coupled to the inner shaft 52.

The frame structure 12 may be detachably coupled to the delivery device 30 in the unexpanded configuration during delivery to the native valve. Expansion of the frame structure 12 to the expanded configuration may detach the frame structure 12 from the delivery device 30.

At least a portion the frame structure 12 may be expanded within at least a portion of the deployed anchor 15 to anchor the frame structure 12 to the native valve. For example, the anchor 15 may be deployed such that it captures one or more structures therein, for example one or more chordae tendineae and/or one or more valve leaflets. Expansion of the frame structure 12, or a portion thereof, within the anchor 15 may compress the captured structures between the frame structure 12 and the anchor 15 to anchor the frame structure 12 in place.

The outer sheath 50 may be steerable as described herein. The outer sheath assembly 50 may be steered through the native valve such that the distal end is positioned on below the native valve and the outer sheath 50 is positioned above the native valve.

The anchor elements 15 may be pushed through the anchor delivery retaining element 242 by a proximal pusher arm disposed within the lumen of the anchor delivery retaining element 242, for example as described in International Patent Application No. PCT/US2020/023671, previously incorporated herein by reference for all purposes. The anchor 15 may be substantially similar to any of the anchors described herein.

The frame structure 12 can be maintained in the delivery configuration by radial constriction from the outer sheath 50.

The anchor 15 may be detachably coupled to a proximal or distal portion of the frame structure 12 as described herein. Alternatively, or in combination, the anchor 15 may be detachably coupled to the delivery device 30 in the delivery configuration during delivery to the native valve. For example, the proximal end of the anchor 15 may be detachably coupled to proximal anchor pushing elements 244 threaded through a lumen of the inner shaft 52 of the delivery device 30. The proximal end of the anchor 15 may be detachably coupled to the proximal anchor pushing element 244 of the delivery device 30 by an attachment element, e.g., an anchor releasing element 243. Alternatively, or in combination, the proximal end of the anchor 15 may be detachably coupled to the proximal anchor pushing element 244 of the delivery device 30 by a weak adhesive.

The anchor 15 may be wrapped around the native chordae tendineae 40 in order to lock the valve prosthesis 10 in place as described herein. The frame structure 10 may be released from the delivery device 30 as described herein. The delivery device 30 may then be retracted to leave the valve prosthesis 10 in place.

The valve prosthesis 10 may be positioned within the native valve as described herein.

In some embodiments, the anchor 15 may comprise a wire or band. In some embodiments, the anchor 15 may comprise a bent wire or band, a coiled wire or band, a helical wire or band, or a spiral wire or band in the deployed configuration. In various embodiments, the wire or band may have a bent, coiled, helical, or spiral shape in the deployed configuration. In various embodiments, the wire or band may be elongated, rather than bent/coil/helical/spiral-shaped—in the delivery configuration. In various embodiments, a portion of the wire or band may have a bent, coiled, helical, or spiral shape.

The anchor 15 may comprise a spiral band or wire. The anchor 15 may be configured to anchor the frame structure 12 to the native valve when the frame structure 12 is in the expanded configuration adjacent the native valve. The frame structure 12 may be configured to be actuated from the unexpanded configuration to the expanded configuration adjacent a native valve in a patient. The anchor 15 may be configured to be fully advanced from a first side of a native valve in a patient (e.g., an atrial side) to a second side of the native valve (e.g., into a ventricle of the heart) and anchor the frame structure 12 to the native valve when the frame structure 12 is in the expanded configuration adjacent the native valve as described herein.

The anchor 15 may comprise a spiral band having a free end 22. The other end of the anchor 15 may be slidably coupled to the top (proximal end) or bottom (distal end) of the frame structure 12 as described herein. Alternatively, or in combination, the other end of the anchor 15 may not be attached to the frame structure 12 as described herein. The free end 22 of the anchor 15 may facilitate capturing of the valve leaflets and/or chordal tendineae within the sweep of the free end during rotation as described herein. During rotation of the anchor 15, the leaflets and/or chordae tendineae may be captured by the free end 22 and trapped between the valve frame structure 12 and an interior surface of the anchor 15.

In some embodiments, the free end 22 may comprise an atraumatic tip to avoid reduce risk of injury to the native valve tissue and leaflets. For example, the free end may comprise a blunt end, a ball tip, a curved tip (e.g., J-tip or pigtail), or other atraumatic shapes. Alternatively, the free end 22 may be configured for piercing tissue.

The free end 22 of the anchor 15 may optionally deployed around one or more structures on the second side of the native valve such that the one or more structures (e.g., chordae, leaflets, or annulus) are pulled radially inwards towards the longitudinal axis of the anchor 15 and/or towards the longitudinal axis of the delivery device 30. The anchor 15 and/or free end 22 may be configured such that minimal torque is applied to the one or more structures. Alternatively, or in combination, the spiral band and/or free end 22 may be configured such that the one or more structures are not pulled or rotated, or are minimally pulled or rotated, during deployment of the anchor 15. For example, the anchor 15 may comprise one or more spaces between loops of the anchor 15 which facilitate movement of the captured tissue (e.g., chordae and/or leaflets) from the free end 22 to the center of the anchor structure with little or no torque, tension, and/or rotation of the structures during deployment of the anchor 15 as described herein. The one or more structures may retain or nearly retain their normal anatomical position when the anchor 15 is fully deployed.

The anchor 15 may comprise a delivery (e.g., an elongated) configuration and a deployed configuration. The anchor 15 may be configured to be actuated from the delivery configuration to the deployed configuration adjacent a native valve in a patient. In various embodiments, the anchor 15 may have a generally spiral shape in the deployed configuration. In various embodiments, the band may be elongated—rather than spiral-shaped—in the delivery configuration. For example, the anchor 15 may be elongated into a straight shape within the anchor retaining elements 242. In various embodiments, a portion of the anchor 15 may have a spiral shape. In various embodiments, a substantial portion of the anchor 15 may have a spiral shape. In various embodiments, the anchor 15 may be formed as a flat spiral (in the deployed configuration) whereby the loops generally are positioned within the same plane (the plane being parallel to a longitudinal axis).

At least a portion of the anchor 15 may be formed of a material having sufficient rigidity to hold a predetermined shape. At least a portion of the anchor 15 may, for example, be formed of a shape memory material (e.g., NiTi). In some cases, at least a portion of the anchor 15 is made of material other than a shape memory material. It may be desirable for at least an end portion (e.g., free end 22) to be relatively rigid such that it can exert a force to move chordal tendineae, while still retaining flexibility to be collapsed within a delivery device. In various embodiments, the end portion only needs sufficient rigidity to hold its shape and will deform under a load. For example, the end portion may be configured with a similar rigidity to a guidewire, or slightly stiffer.

The delivery device 30 may be substantially similar to any of the delivery devices described herein. For example, the delivery device 30 may comprise an outer sheath in which the anchor 15 may be disposed, for example in an elongated or undeployed delivery configuration as described herein. Alternatively, or in combination, the delivery device 30 may comprise an inner shaft, for example disposed within the lumen of the outer sheath. The anchor 15 and/or frame structure 12 may be coupled to the inner shaft as described herein. The delivery device 30 may comprise a guidewire as described herein. The delivery device 30 may be configured to deliver the anchor 15 to the native valve as described herein. The manner of implanting valve device 10 may be substantially similar to any of the methods described herein.

Interaction of the frame structure with the one or more loops of the anchor may create opposing forces therebetween that provide mechanical leverage for anchoring the frame structure to the one or more anatomical structures. In some embodiments, the one or more loops may comprise at least 360 degrees of rotation when deployed such that the loops wrap around one another and provide additional mechanical leverage against the frame structure in order to facilitate anchoring of the frame structure as described herein. Additional loops or partial loops may provide additional mechanical strength and/or leverage.

The anchor 15 may comprise a spiral wire or band. The anchor 15 may comprise a plurality of spiral wires or bands as described herein.

In some embodiments, the delivery device 30 may be configured to carry the anchor 15 in an undeployed configuration and deploy the anchor 15 into a deployed configuration as the desired location as described herein.

In some embodiments, the anchor 15 may comprise one or more locking mechanisms configured to maintain the anchor 15 in the deployed configuration. The one or more locking mechanisms may be any of the locking mechanisms described herein or understood by one of ordinary skill in the art from the description herein. In various embodiments, one or more loops may be nested with each other when the anchor is in the deployed configuration.

Additional description for the anchor and other similar anchors usable in the embodiments described herein may be found in U.S. patent applications Ser. No. 16/546,901, 16/594,946, 16/723,537, and International Patent Applications No. PCT/US2019/047542, PCT/US2019/055049, PCT/US2019/057082, PCT/US2019/068088, PCT/US2020/023671, PCT/US2020/027744, and PCT/US2020/058413, which are incorporated herein by reference in their entireties for all purposes.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A system for treating a diseased native valve in a patient, the system comprising:
a frame structure having a compressed configuration and an expanded configuration wherein the frame structure comprises one or more anchor retaining elements disposed along one or more lateral sides of the frame structure;
one or more anchor elements having a free end and configured to be slidably disposed within the one or more anchor retaining elements of the frame structure;
wherein the anchor is configured to be fully disposed within the anchor retaining elements in a delivery configuration,
wherein the anchor is configured to be at least partially external to the anchor retaining elements in a deployed configuration, and
wherein the anchor is configured to be advanced from the delivery configuration to the deployed configuration when the frame structure is in the expanded configuration adjacent the diseased native valve.

2. The system of claim 1, further comprising a delivery device, the delivery device comprising an outer sheath and an inner sheath extendable from the outer sheath, the inner sheath having a lumen through which proximal anchor pusher elements are threaded, wherein the proximal anchor pusher elements are connected to one or more anchors by one or more anchor releasing elements.

3. (canceled)

4. The system of claim 2, wherein the outer sheath has a lumen and is configured to maintain the inner sheath in an elongated configuration when positioned within the lumen.

5. (canceled)

6. The system of claim 2, wherein the inner sheath is steerable.

7. The system of claim 2, wherein a proximal end of the one or more anchors is detachably coupled to the one or more proximal anchor pusher elements by one or more anchor releasing elements during delivery to the native valve.

8. The system of claim 7, wherein the one or more anchors are configured to be elongated when disposed within the anchor retaining elements.

9. The system of claim 8, wherein advancement of the one or more anchor pusher elements from the lumen of the inner shaft into the anchor retaining elements actuates the one or more anchors into the deployed configuration.

10. The system of claim 8, wherein the one or more anchors are maintained in the elongated configuration by radial constriction from the anchor retaining elements and wherein advancement of the one or more proximal anchor pusher elements out of the lumen of the inner shaft actuates the anchor into the deployed configuration.

11. The system of claim 8, wherein the proximal end of the anchor is detachably coupled to the and wherein retraction of the one or more proximal anchor pusher elements away from the proximal end of the one or more anchors detaches the one or more anchors from the one or more proximal anchor pusher elements.

12. The system of claim 2, wherein the one or more anchors is configured to lock into the one or more anchor retaining elements after actuation of the one or more anchor releasing elements.

13. (canceled)

14. The system of claim 1, wherein the frame structure is detachably coupled to the delivery device in the compressed configuration during delivery to the native valve.

15. The system of claim 14, wherein expansion of the frame structure to the expanded configuration detaches the frame structure from the delivery device.

16. (canceled)

17. The system of claim 1, wherein the free end comprises an atraumatic tip.

18. (canceled)

19. (canceled)

20. The system of claim 1, wherein the one or more anchor elements comprise a helical wire or band.

21. The system of claim 20, wherein the one or more anchor elements comprise a first portion comprising the helical wire or band, and a second portion comprising a straight wire or band.

22. (canceled)

23. (canceled)

24. The system of claim 1, wherein the compressed configuration is sized and dimensioned for percutaneous insertion and the expanded configuration is sized and dimensioned for implantation in the native valve of the patient.

25. The system of claim 1, wherein the frame structure comprises a first and second opposite ends, the first end extending above a native valve and the second end extending below the native valve when the frame structure is anchored to the native valve.

26. The system of claim 23, wherein the frame structure sits below the native valve when the frame structure is anchored to the native valve.

27. The system of claim 1, wherein the frame structure comprises an expandable stent.

28. The system of claim 1, wherein the expanded configuration is a generally tubular expanded shape.

29. The system of claim 1, wherein the frame structure comprises an expanded outer periphery in the expanded configuration and a compressed outer periphery when subject to an external radial force in the compressed configuration, wherein the compressed outer periphery is smaller in diameter than the expanded outer periphery.

30. (canceled)

31. The system of claim 1, wherein the frame structure is self-expanding.

32.-112. (canceled)
